(19) United States
(12) Patent Application Publication
(10) Pub. No.: US 2025/0275720 A1
(43) Pub. Date: Sep. 4, 2025

(54) SYSTEM AND METHOD FOR METABOLIC SENSING USING WEARABLE DEVICE

(71) Applicant: Samsung Electronics Co., Ltd., Gyeonggi-do (KR)

(72) Inventors: Shailabh KUMAR, Pasadena, CA (US); Yibing Michelle WANG, Pasadena, CA (US); Radwanul Hasan SIDDIQUE, Monrovia, CA (US); Sang Kyu KIM, Gyeonggi-do (KR)

(21) Appl. No.: 19/057,325

(22) Filed: Feb. 19, 2025

Related U.S. Application Data
(60) Provisional application No. 63/559,335, filed on Feb. 29, 2024.

(51) Int. Cl. A61B 5/00; A61B 5/0205; A61B 5/11; A61B 5/145; A61B 5/318

(52) U.S. Cl. CPC A61B 5/4866; A61B 5/02055; A61B 5/1118; A61B 5/14532; A61B 5/14542; A61B 5/318; A61B 5/6802; A61B 5/7264; A61B 2562/0219

(57) ABSTRACT

A system and a method are disclosed for metabolic sensing using a wearable device. A method includes receiving first sensor data from a first sensor worn by a user; receiving second sensor data from a second sensor worn by the user, the second sensor being a different type of sensor than the first sensor; generating based at least in part on the first sensor data and the second sensor data, an MH indicator, the MH indicator corresponding to the first sensor data, the second sensor data, and third sensor data of a third sensor that is different from the first and second sensors; and providing the MH indicator to the user.

### CROSS-REFERENCE TO RELATED APPLICATION

This application claims the priority benefit under 35 U.S.C. § 119(e) of U.S. Provisional Application No. 63/559,335, filed on Feb. 29, 2024, the disclosure of which is incorporated by reference in its entirety as if fully set forth herein.

### TECHNICAL FIELD

The disclosure generally relates to health monitoring using a wearable device. More particularly, the subject matter disclosed herein relates to improvements to measurement of metabolic health (MH), metabolic fitness (MF), and metabolic biomarkers (MBs) provided by multisensory wearable devices.

### SUMMARY

Metabolic syndrome is a chronic condition that is generally identified when someone has three or more of the following risk factors:
high blood pressure, e.g., blood pressure of 130/80 or higher;
high blood sugar, e.g., a fasting glucose level of 100 mg/dL or higher;
high triglycerides, e.g., triglyceride levels of 150 mg/dL or higher;
low high-density lipoprotein (HDL) cholesterol, e.g., HDL cholesterol levels below 40 mg/dL for men and below 50 mg/dL for women; and/or
large waist circumference, e.g., 40 inches or larger for men and 35 inches or larger for women.

Metabolic syndrome increases the risk of heart disease, diabetes, stroke, cardiovascular diseases, and other health problems.

To combat metabolic syndrome, metabolic monitoring may be utilized for diabetes patients or people at risk of metabolic diseases, for weight loss, cardiovascular health, and aging for general population.

In addition to combating metabolic syndrome, metabolic monitoring may be utilized for fitness, nutrition, and recovery guidance for people in good health, e.g., athletes.

For example, quantitative measurement of metabolic health on wearable devices can be provided based on metabolic rate (MR), MF, and/or MB monitoring.

MR may be defined as a rate of energy consumption (e.g., a basal metabolic rate (BMR), a resting metabolic rate (RMR), or an active metabolic rate (AMR)) and is often used for calorie and body mass index (BMI) management

MF generally refers to how well a person can produce energy, use nutrients, and regulate hormones. For example, MF may be measure individual fitness compared to mean performance in a person's age group (e.g., metabolic age, Glucose variation index, Lactate threshold, etc.), and may be useful for measuring rate of metabolic aging.

MB monitoring is the process of regularly measuring MBs to assess a person's metabolic health. MBs such as the concentration of molecules in blood including glucose, lactate, lipids (cholesterol, triglycerides), insulin, etc., are indicators of health, such as blood sugar, blood pressure, and cholesterol levels, and as such may be useful for monitoring risk of metabolic and cardiovascular diseases.

Current wearables provide an equation-based calculation of BMR, and then calculate active metabolism (i.e., calories burned) using estimation of activity from wearable motion trackers. However, evidence suggests that both basal metabolism and active metabolism calculated using these methods are extremely error-prone.

In addition to inaccuracies with MR and MF predictions, current wearables cannot non-invasively measure MBs such as glucose, lactate, lipids at the moment. While non-invasive wearable sensors for measuring MBs have been attempted, so far these devices are not accurate enough.

Minimally invasive wearable sensors for MBs, such as glucose sensors, need replacement every 14 days, and therefore, are not convenient for use.

To address these types of issues, systems and methods are described herein for providing accurate measurement of metabolic health, fitness, and biomarkers using multisensory wearable devices.

Accordingly, an aspect of the disclosure is to provide a novel algorithm for accurate measurement of metabolic health, fitness, and biomarkers using multisensory wearable devices.

Another aspect of the disclosure is to provide a combination of hardware sensors to generate data for developing an algorithm for accurate measurement of MH, MF, and MBs.

Another aspect of the disclosure is to provide a training dataset, which may be generated as a result of various wearable sensors operating simultaneously.

Another aspect of the disclosure is to provide an MF algorithm with artificial intelligence (AI)/machine learning (ML) processes trained on sensor input from multiple wearable sensors.

Another aspect of the disclosure is to provide a method and system for sensor-free measurement of MBs (e.g., glucose, lactate, etc.) by utilizing an AI/ML-based algorithm.

In accordance with an aspect of the disclosure, more accurate MR and fitness prediction can be performed.

In accordance with another aspect of the disclosure, unlike current wearables devices, a wearable device according to an embodiment of the disclosure may measure MBs such as glucose, lactate, lipids, etc., non-invasively, without the need for sensor replacement.

In an embodiment, a method is provided for metabolic sensing. The method includes receiving first sensor data from a first sensor worn by a user; receiving second sensor data from a second sensor worn by the user, the second sensor being a different type of sensor than the first sensor; generating based at least in part on the first sensor data and the second sensor data, an MH indicator, the MH indicator corresponding to the first sensor data, the second sensor data, and third sensor data of a third sensor that is different from the first and second sensors; and providing the MH indicator to the user.

In an embodiment, a system is provided for metabolic sensing. The system includes a first sensor worn by a user; a second sensor worn by the user, the second sensor being a different type of sensor than the first sensor; and a processor configured to receive first sensor data from the first sensor, receive second sensor data from the second sensor, generate based at least in part on the first sensor data and the second sensor data, an MH indicator, the MH indicator corresponding to the first sensor data, the second sensor data, and third sensor data of a third sensor that is different from the first and second sensors, and provide the MH indicator to the user.

### BRIEF DESCRIPTION OF THE DRAWING

In the following section, the aspects of the subject matter disclosed herein will be described with reference to exemplary embodiments illustrated in the figures, in which:

FIG. 1 illustrates a general overview an operation method, according to an embodiment;

FIG. 2 illustrates a flow diagram for algorithm development, according to an embodiment;

FIG. 3 illustrates examples of algorithm outputs from a wearable device, according to an embodiment;

FIG. 4 illustrates a flow diagram for algorithm development, according to an embodiment;

FIG. 5 is a flow chart illustrating a method according to an embodiment; and

FIG. 6 is a block diagram of an electronic device in a network environment, according to an embodiment.

### DETAILED DESCRIPTION

In the following detailed description, numerous specific details are set forth in order to provide a thorough understanding of the disclosure. It will be understood, however, by those skilled in the art that the disclosed aspects may be practiced without these specific details. In other instances, well-known methods, procedures, components and circuits have not been described in detail to not obscure the subject matter disclosed herein.

Reference throughout this specification to “one embodiment” or “an embodiment” means that a particular feature, structure, or characteristic described in connection with the embodiment may be included in at least one embodiment disclosed herein. Thus, the appearances of the phrases “in one embodiment” or “in an embodiment” or “according to one embodiment” (or other phrases having similar import) in various places throughout this specification may not necessarily all be referring to the same embodiment. Furthermore, the particular features, structures or characteristics may be combined in any suitable manner in one or more embodiments. In this regard, as used herein, the word “exemplary” means “serving as an example, instance, or illustration.” Any embodiment described herein as “exemplary” is not to be construed as necessarily preferred or advantageous over other embodiments. Additionally, the particular features, structures, or characteristics may be combined in any suitable manner in one or more embodiments. Also, depending on the context of discussion herein, a singular term may include the corresponding plural forms and a plural term may include the corresponding singular form. Similarly, a hyphenated term (e.g., “two-dimensional,” “pre-determined,” “pixel-specific,” etc.) may be occasionally interchangeably used with a corresponding non-hyphenated version (e.g., “two dimensional,” “predetermined,” “pixel specific,” etc.), and a capitalized entry (e.g., “Counter Clock,” “Row Select,” “PIXOUT,” etc.) may be interchangeably used with a corresponding non-capitalized version (e.g., “counter clock,” “row select,” “pixout,” etc.). Such occasional interchangeable uses shall not be considered inconsistent with each other.

Also, depending on the context of discussion herein, a singular term may include the corresponding plural forms and a plural term may include the corresponding singular form. It is further noted that various figures (including component diagrams) shown and discussed herein are for illustrative purpose only, and are not drawn to scale. For example, the dimensions of some of the elements may be exaggerated relative to other elements for clarity. Further, if considered appropriate, reference numerals have been repeated among the figures to indicate corresponding and/or analogous elements.

The terminology used herein is for the purpose of describing some example embodiments only and is not intended to be limiting of the claimed subject matter. As used herein, the singular forms “a,” “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

It will be understood that when an element or layer is referred to as being on, “connected to” or “coupled to” another element or layer, it can be directly on, connected or coupled to the other element or layer or intervening elements or layers may be present. In contrast, when an element is referred to as being “directly on,” “directly connected to” or “directly coupled to” another element or layer, there are no intervening elements or layers present. Like numerals refer to like elements throughout. As used herein, the term “and/or” includes any and all combinations of one or more of the associated listed items.

The terms “first,” “second,” etc., as used herein, are used as labels for nouns that they precede, and do not imply any type of ordering (e.g., spatial, temporal, logical, etc.) unless explicitly defined as such. Furthermore, the same reference numerals may be used across two or more figures to refer to parts, components, blocks, circuits, units, or modules having the same or similar functionality. Such usage is, however, for simplicity of illustration and ease of discussion only; it does not imply that the construction or architectural details of such components or units are the same across all embodiments or such commonly-referenced parts/modules are the only way to implement some of the example embodiments disclosed herein.

Unless otherwise defined, all terms (including technical and scientific terms) used herein have the same meaning as commonly understood by one of ordinary skill in the art to which this subject matter belongs. It will be further understood that terms, such as those defined in commonly used dictionaries, should be interpreted as having a meaning that is consistent with their meaning in the context of the relevant art and will not be interpreted in an idealized or overly formal sense unless expressly so defined herein.

As used herein, the term “module” refers to any combination of software, firmware and/or hardware configured to provide the functionality described herein in connection with a module. For example, software may be embodied as a software package, code and/or instruction set or instructions, and the term “hardware,” as used in any implementation described herein, may include, for example, singly or in any combination, an assembly, hardwired circuitry, programmable circuitry, state machine circuitry, and/or firmware that stores instructions executed by programmable circuitry. The modules may, collectively or individually, be embodied as circuitry that forms part of a larger system, for example, but not limited to, an integrated circuit (IC), system on-a-chip (SoC), an assembly, and so forth.

Herein, a wearable device may refer to a smartwatch or a fitness tracking device, e.g., which may be worn around the wrist or as a ring.

As described above, embodiments of the disclosure are directed to providing accurate measurement of MH, MF, and MBs using multisensory wearable devices. More specifically, new algorithms are provided for predicting MH of wearers using multi-sensor information from wearable devices, where the algorithms utilize the wearable devices or sets of devices with multiple sensors. Examples of the sensors may include, but are not limited to, photoplethysmography (PPG) sensors, inertial measurement units (IMUs), electrocardiogram (ECG) sensors, temperature sensors, multispectral sensors, polarization sensors, depth/3-dimensional (3D) sensors, biomolecular sensors, electrochemical sensors, piezoelectric sensors, field-effect transistor (FET) sensors, immunosensors, nanomaterial-based sensors, etc.

Accordingly, using the multiple sensors, wearable sensor output in the form of heart rate (HR), HR variability (HRV), oxygen saturation (SpO2), steps taken, activity, glucose level, lactate level, skin temperature, core body temperature, etc., may be used to build and train the algorithms.

The algorithms may use AI neural network (NN)-based ML tools for generating output.

The algorithms may use clinical laboratory tests or commercial sensor derived health data as ground truth for training, validating, and/or testing an NN model.

As PPG sensors, IMUs, ECG sensors, multispectral sensors, polarization sensors, depth/3-dimensional (3D) sensors, and temperature sensors are already often included on wearable devices, such as smart watches, according to an embodiment, the algorithms may be use predict MH, corresponding to the data previously collected by the biomolecular sensors, electrochemical sensors, piezoelectric sensors, field-effect transistor (FET) sensors, immunosensors, nanomaterial-based sensors, etc. For example, as biomolecular sensors are often invasive, need frequent replacement, and are generally inconvenient to use, the algorithms may be use determine MH using the sensors already included in a wearable device without having to continually use biomolecular sensors.

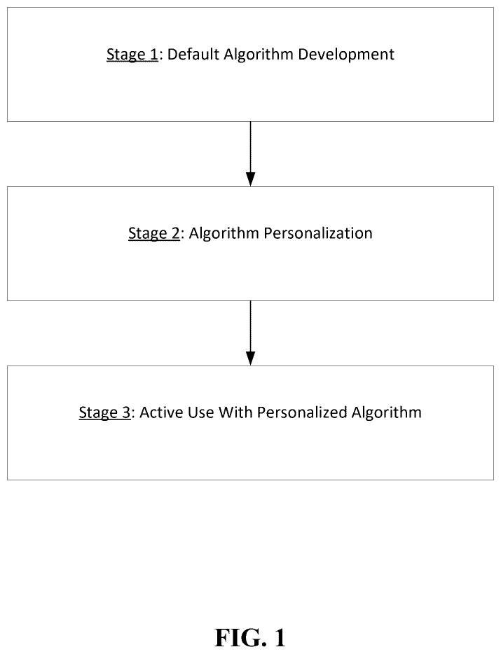

FIG. 1 illustrates a general overview an operation method, according to an embodiment.

Referring to FIG. 1, the operation method includes three generalized stages:
Stage 1: Default Algorithm Development;
Stage 2: Algorithm Personalization; and
Stage 3: Active Use With Personalized Algorithm.

In stage 1, a default algorithm is developed using relatively large amounts of sensor data obtained during a testing period, e.g., months, from multiple users. For example, a large number of users, e.g., 10,000 users, will wear PPG sensors, IMUs, ECG sensors, temperature sensors, and biomolecular sensors for the testing period. The data collected from the sensors, e.g., HR, HRV, SpO2, steps taken, activity, glucose level, lactate level, skin temperature, core body temperature, etc., may be inputs for the default algorithm development.

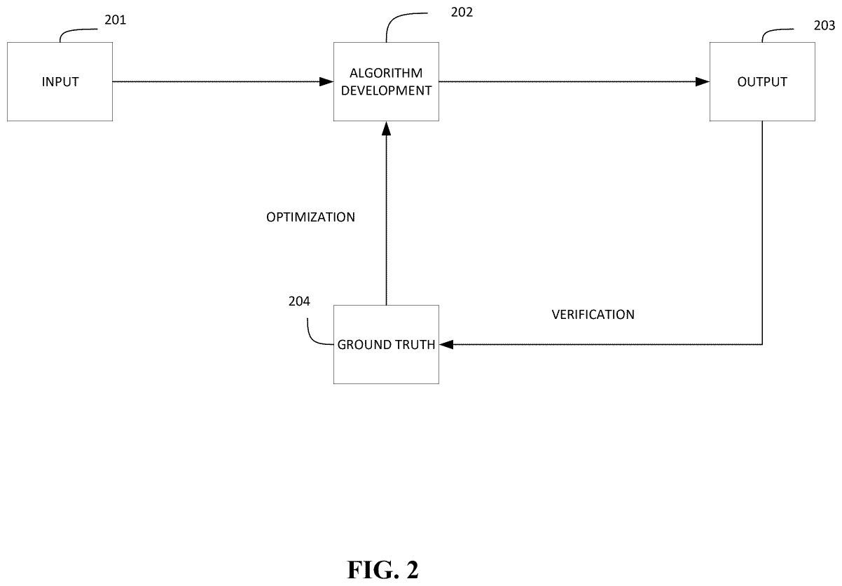

FIG. 2 illustrates a flow diagram for algorithm development, according to an embodiment.

Referring to FIG. 2, in step 201, input from the various sensors is input to the system. For example, data collected from the sensors, e.g., HR, HRV, SpO2, steps taken, activity, glucose level, lactate level, skin temperature, and core body temperature, may be used inputs.

In step 202, an algorithm is developed from the input data. The algorithm may use AI NN-based ML tools for generating output.

In step 203, output is generated from the algorithm. The algorithm may provide an output of algorithm calculated BMR and/or AMR. The algorithm may provide an MF indicator, e.g., metabolic age, glucose variation index (GVI), etc., as output. The algorithm may provide an MB, e.g., glucose, lactate, triglyceride, or insulin concentration in blood, as output.

The MH output in step 203 may include at least one of MR, MF, or MB. The MR may include BMR and/or AMR. The MF may include metabolic age, GVI, etc. The MB may include blood levels of glucose, lactate, triglycerides, insulin, etc. Accordingly, MH output in step 203 may be a function of MR, MF, and MB as shown in Equation (1) below.

$$MH ⊇ ( MR , MF , MB ) = f ( HR , HRV , {SpO}_{2} , Steps , Activity , {T}_{body} , glucose , lactate , Time \qquad ( 1 )$$

In step 204, verification may be performed on the output by comparison to ground truth data. The algorithms may use clinical laboratory tests or commercial sensor derived health data as ground truth for training, validating, and/or testing the NN model.

Thereafter, the results of the verification can be fed back into the system for optimization of the algorithm in step 202.

Referring again to FIG. 1, in stage 2, algorithm personalization may be performed. More specifically, after the default algorithm is developed in stage 1, the default algorithm may be further refined according to an individual user's MH.

For example, for a shorter testing period than in stage 1, e.g., weeks, a user will wear a PPG sensor, an IMU, an ECG sensor, a temperature sensor, and a biomolecular sensor for the testing period. As described above, since a wearable device, such as a smart watch, may include a PPG sensor, an IMU, an ECG sensor, and a temperature sensor, the user may only have to wear an additional biomolecular sensor for testing period. The data collected from the sensors, e.g., HR, HRV, SpO2, steps taken, activity, glucose level, lactate level, skin temperature, core body temperature, etc., may be used as inputs, similar to FIG. 2, for refining the default algorithm for the individual user.

In stage 3, after the default algorithm has been refined for the individual user, the user is able to monitor MH wearing only the wearable device, without having to wear an additional biomolecular sensor. More specifically, using the wearable device including a PPG sensor, an IMU, an ECG sensor, and a temperature sensor, the data collected from these sensors, e.g., HR, HRV, SpO2, steps taken, activity, skin temperature, core body temperature, etc., may be used as inputs for the refined algorithm to determine a corresponding MH output.

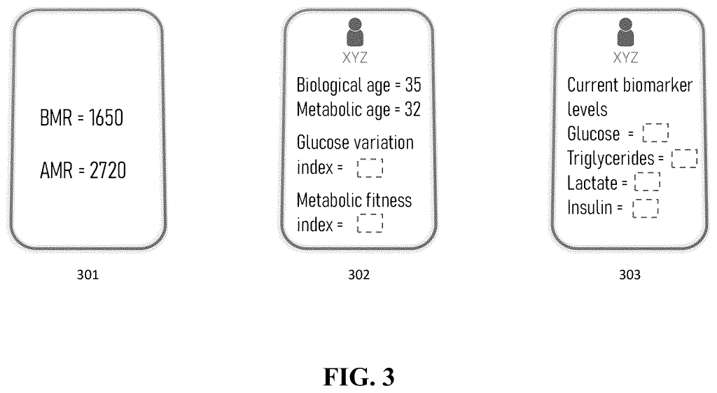

FIG. 3 illustrates examples of algorithm outputs from a wearable device, according to an embodiment.

Referring to FIG. 3, in screen 301, the algorithm output includes a calculated BMR and AMR. In screen, 302 the algorithm output includes a biological age, a calculated metabolic age, a calculated GVI value, and a calculated MF index value. In screen 303, the algorithm includes current MB level, i.e., glucose level, triglyceride level, lactate level, and insulin level. Accordingly, a user can easily monitor their MH without having to continually wear an additional biomolecular sensor.

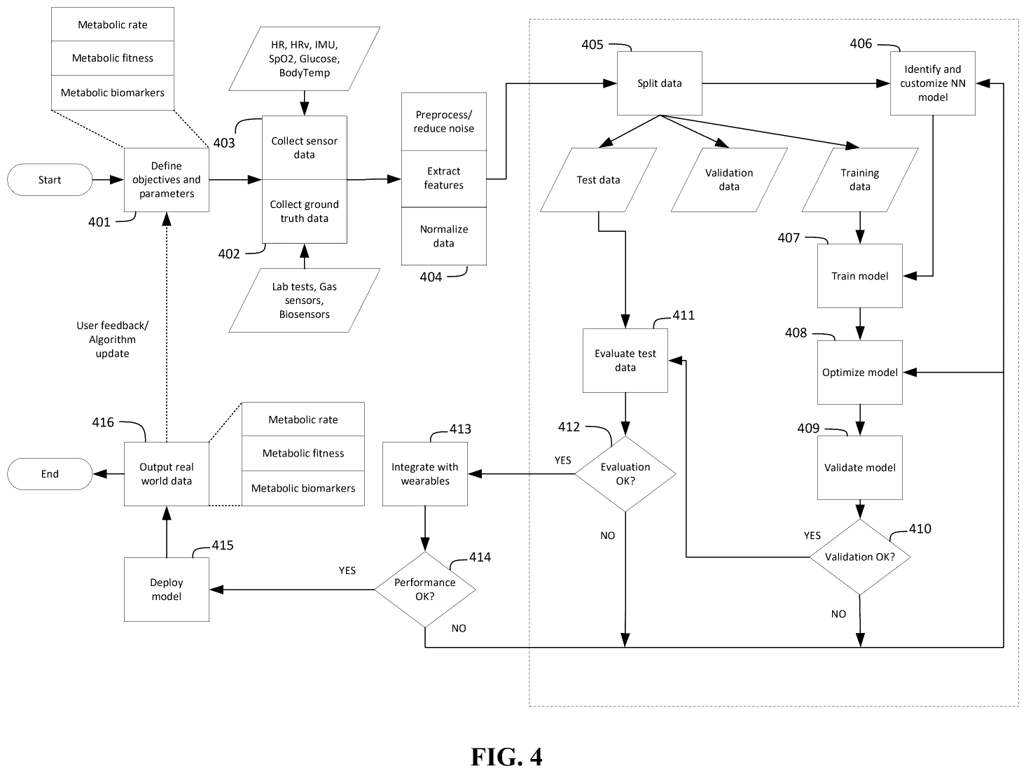

FIG. 4 illustrates a flow diagram for algorithm development, according to an embodiment. For example, FIG. 4 illustrates detailed operations that may be performed during step 202 of FIG. 2.

Referring to FIG. 4, in step 401, system objectives and parameters are defined. For example, the objectives may be defined as MH information including MR, MF, and MBs, based on system parameters including HR, HRV, SpO2, steps taken, activity, glucose level, lactate level, skin temperature, and core body temperature.

In step 402, ground truth data is collected, e.g., from lab tests, gas sensors, and/or biosensors.

In step 403, sensor data is collected, e.g., from wearable PPG sensors, IMUs, ECG sensors, temperature sensors, and biomolecular sensors.

In step 404, the collected data is processed for NN input. More specifically, processing the collected data may include pre-processing to reduce noise, feature extraction, data normalization.

In step 405, the processed data is provided to an NN, which splits the processed data into test data, validation data, and training data.

In step 406, based on the received processed data, an NN model is identified and customized accordingly. For example, the NN model may include but are not limited to convolutional neural networks (CNNs), recurrent neural networks (RNNs), transformer-based architectures, autoencoders, self-supervised learning models, reinforcement learning models, or hybrid and unique combinations of these architectures for the sensor data.

In step 407, the NN trains the NN model using a major fraction of sensor data anointed as “the training dataset”.

In step 408, the NN model is optimized based on the training performed over several iterations.

In step 409, the optimized NN model is validated using a minor fraction of sensor data that was not used in the training step (termed as “validation dataset”).

Although not illustrated in FIG. 4 for simplicity, multiple cycles of training and validation may be performed (called “epochs”) to finalize model architecture and performance.

If it is determined that the optimized NN model is successfully validated in step 410, then the sensor test data is evaluated using the optimized NN model in step 411. However, if it is determined that the optimized NN model is not successfully validated in step 410, then the procedure returns to step 408 to re-optimize the NN model in view of the unsuccessful validation.

If it is determined that the evaluation of the test data with the optimized NN model is successful in step 412, then the optimized NN model is integrated with a wearable device, e.g., a smart watch, in step 413. However, if it is determined that the evaluation of the test data with the optimized NN model is unsuccessful in step 412, then the procedure returns to step 408 or 406 to re-optimize the NN model or identify and customize a new NN model in view of the unsuccessful evaluation.

If it is determined that performance of the wearable device integrated with the optimized NN model is satisfactory in step 412, e.g., meets predetermined power consumption and speed benchmarks, then the optimized NN model is deployed for use with wearable devices. However, if it is determined that performance of the wearable device integrated with the optimized NN model is not satisfactory in step 412, then the procedure returns to step 408 or 406 to re-optimize the NN model or identify and customize a new NN model in view of the unsuccessful performance. This may also include steps for implementing low-power and edge AI solutions such as quantized NN models, model pruning, and sparse networks, which often require less memory to run.

In step 416, the deployed wearable device integrated with the optimized NN model is actively used to output MH readings (e.g., MR, MF, and/or MBs), based on acquired sensor data.

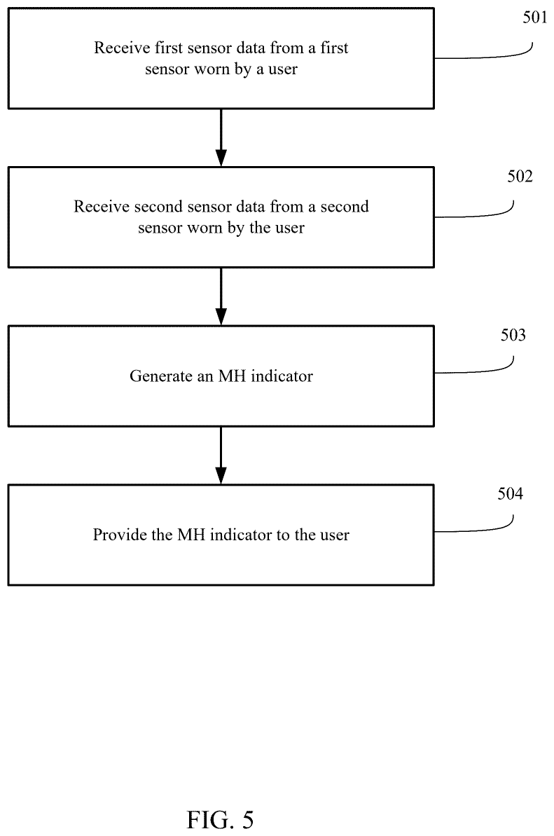

FIG. 5 is a flow chart illustrating a method according to an embodiment.

Referring to FIG. 5, in step 501, an electronic device, such as a smart watch, receives first sensor data from a first sensor worn by a user.

In step 502, the electronic device receives second sensor data from a second sensor worn by the user. The second sensor is a different type of sensor than the first sensor.

For example, the first sensor or the second sensor may include at least one of a PPG sensor, an IMU, an ECG sensor, a multispectral sensor, a polarization sensor, a 3D sensor, or a temperature sensor.

In step 503, the electronic device generates, based at least in part on the first sensor data and the second sensor data, an MH indicator. The MH indicator corresponds to the first sensor data, the second sensor data, and third sensor data of a third sensor that is different from the first and second sensors.

As illustrated in FIG. 4, the electronic device may enter the first sensor data and the second sensor data into an algorithm for outputting an MH indicator. The MH indicator corresponds to the first sensor data, the second sensor data, and third sensor data of a third sensor not being worn by the user. For example, the third sensor may include at least one of a biomolecular sensor, an electrochemical sensor, a piezoelectric sensor, a FET sensor, an immunosensor, or a nanomaterial-based sensor.

In step 504, the electronic device provides the MH indicator to the user. For example, the electronic device displays screen 301, 302, or 303, as illustrated in FIG. 3.

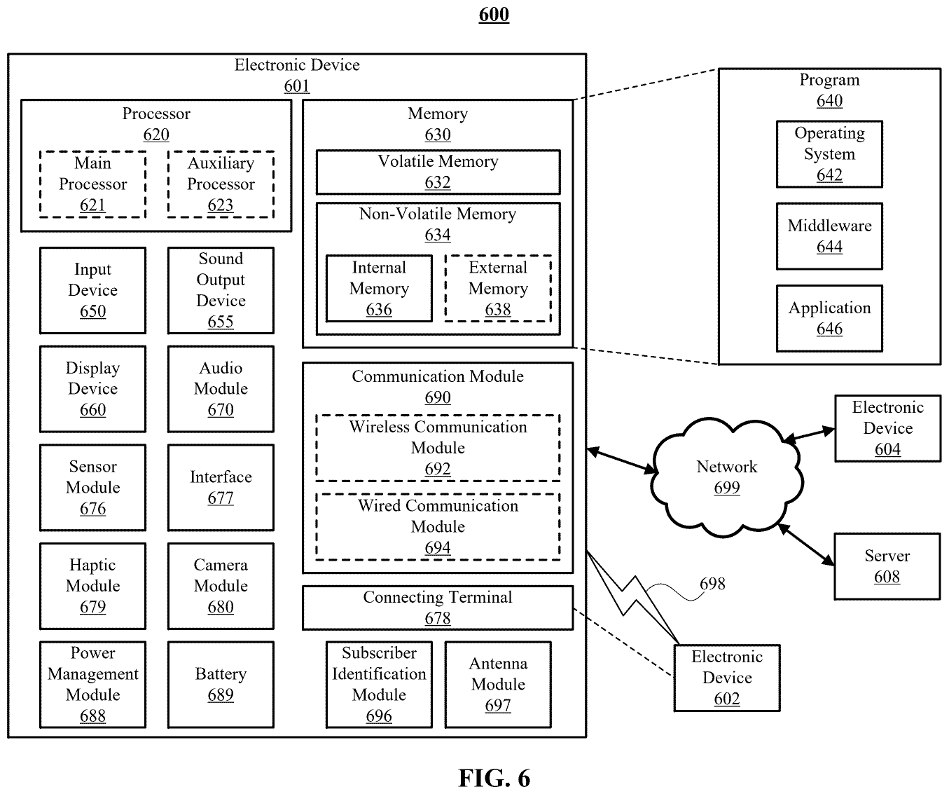

FIG. 6 is a block diagram of an electronic device in a network environment 600, according to an embodiment.

Referring to FIG. 6, an electronic device 601, e.g., a wearable device such as a smart watch, in a network environment 600 may communicate with an electronic device 602 via a first network 698 (e.g., a short-range wireless communication network), or an electronic device 604 or a server 608 via a second network 699 (e.g., a long-range wireless communication network). The electronic device 601 may communicate with the electronic device 604 via the server 608. The electronic device 601 may include a processor 620, a memory 630, an input device 650, a sound output device 655, a display device 660, an audio module 670, a sensor module 676, an interface 677, a haptic module 679, a camera module 680, a power management module 688, a battery 689, a communication module 690, a subscriber identification module (SIM) card 696, or an antenna module 697. In one embodiment, at least one (e.g., the display device 660 or the camera module 680) of the components may be omitted from the electronic device 601, or one or more other components may be added to the electronic device 601. Some of the components may be implemented as a single integrated circuit (IC). For example, the sensor module 676 (e.g., a fingerprint sensor, an iris sensor, or an illuminance sensor) may be embedded in the display device 660 (e.g., a display).

The processor 620 may execute software (e.g., a program 640) to control at least one other component (e.g., a hardware or a software component) of the electronic device 601 coupled with the processor 620 and may perform various data processing or computations.

As at least part of the data processing or computations, the processor 620 may load a command or data received from another component (e.g., the sensor module 676 or the communication module 690) in volatile memory 632, process the command or the data stored in the volatile memory 632, and store resulting data in non-volatile memory 634. The processor 620 may include a main processor 621 (e.g., a central processing unit (CPU) or an application processor (AP)), and an auxiliary processor 623 (e.g., a graphics processing unit (GPU), an image signal processor (ISP), a sensor hub processor, or a communication processor (CP)) that is operable independently from, or in conjunction with, the main processor 621. Additionally or alternatively, the auxiliary processor 623 may be adapted to consume less power than the main processor 621, or execute a particular function. The auxiliary processor 623 may be implemented as being separate from, or a part of, the main processor 621.

The auxiliary processor 623 may control at least some of the functions or states related to at least one component (e.g., the display device 660, the sensor module 676, or the communication module 690) among the components of the electronic device 601, instead of the main processor 621 while the main processor 621 is in an inactive (e.g., sleep) state, or together with the main processor 621 while the main processor 621 is in an active state (e.g., executing an application). The auxiliary processor 623 (e.g., an image signal processor or a communication processor) may be implemented as part of another component (e.g., the camera module 680 or the communication module 690) functionally related to the auxiliary processor 623.

The memory 630 may store various data used by at least one component (e.g., the processor 620 or the sensor module 676) of the electronic device 601. The various data may include, for example, software (e.g., the program 640) and input data or output data for a command related thereto. The memory 630 may include the volatile memory 632 or the non-volatile memory 634. Non-volatile memory 634 may include internal memory 636 and/or external memory 638.

The program 640 may be stored in the memory 630 as software, and may include, for example, an operating system (OS) 642, middleware 644, or an application 646.

The input device 650 may receive a command or data to be used by another component (e.g., the processor 620) of the electronic device 601, from the outside (e.g., a user) of the electronic device 601. The input device 650 may include, for example, a microphone, a mouse, or a keyboard.

The sound output device 655 may output sound signals to the outside of the electronic device 601. The sound output device 655 may include, for example, a speaker or a receiver. The speaker may be used for general purposes, such as playing multimedia or recording, and the receiver may be used for receiving an incoming call. The receiver may be implemented as being separate from, or a part of, the speaker.

The display device 660 may visually provide information to the outside (e.g., a user) of the electronic device 601. The display device 660 may include, for example, a display, a hologram device, or a projector and control circuitry to control a corresponding one of the display, hologram device, and projector. The display device 660 may include touch circuitry adapted to detect a touch, or sensor circuitry (e.g., a pressure sensor) adapted to measure the intensity of force incurred by the touch.

The audio module 670 may convert a sound into an electrical signal and vice versa. The audio module 670 may obtain the sound via the input device 650 or output the sound via the sound output device 655 or a headphone of an external electronic device 602 directly (e.g., wired) or wirelessly coupled with the electronic device 601.

The sensor module 676 may detect an operational state (e.g., power or temperature) of the electronic device 601 or an environmental state (e.g., a state of a user) external to the electronic device 601, and then generate an electrical signal or data value corresponding to the detected state. The sensor module 676 may include, for example, a gesture sensor, a gyro sensor, an atmospheric pressure sensor, a magnetic sensor, an acceleration sensor, a grip sensor, a proximity sensor, a color sensor, an infrared (IR) sensor, a biometric sensor, a temperature sensor, a humidity sensor, or an illuminance sensor. For example, the sensor module 676 may also include a PPG sensor, an IMUs, and an ECG sensor, as described above.

The interface 677 may support one or more specified protocols to be used for the electronic device 601 to be coupled with the external electronic device 602 directly (e.g., wired) or wirelessly. The interface 677 may include, for example, a high-definition multimedia interface (HDMI), a universal serial bus (USB) interface, a secure digital (SD) card interface, or an audio interface.

A connecting terminal 678 may include a connector via which the electronic device 601 may be physically connected with the external electronic device 602. The connecting terminal 678 may include, for example, an HDMI connector, a USB connector, an SD card connector, or an audio connector (e.g., a headphone connector).

The haptic module 679 may convert an electrical signal into a mechanical stimulus (e.g., a vibration or a movement) or an electrical stimulus which may be recognized by a user via tactile sensation or kinesthetic sensation. The haptic module 679 may include, for example, a motor, a piezoelectric element, or an electrical stimulator.

The camera module 680 may capture a still image or moving images. The camera module 680 may include one or more lenses, image sensors, image signal processors, or flashes. The power management module 688 may manage power supplied to the electronic device 601. The power management module 688 may be implemented as at least part of, for example, a power management integrated circuit (PMIC).

The battery 689 may supply power to at least one component of the electronic device 601. The battery 689 may include, for example, a primary cell which is not rechargeable, a secondary cell which is rechargeable, or a fuel cell.

The communication module 690 may support establishing a direct (e.g., wired) communication channel or a wireless communication channel between the electronic device 601 and the external electronic device (e.g., the electronic device 602, the electronic device 604, or the server 608) and performing communication via the established communication channel. The communication module 690 may include one or more communication processors that are operable independently from the processor 620 (e.g., the AP) and supports a direct (e.g., wired) communication or a wireless communication. The communication module 690 may include a wireless communication module 692 (e.g., a cellular communication module, a short-range wireless communication module, or a global navigation satellite system (GNSS) communication module) or a wired communication module 694 (e.g., a local area network (LAN) communication module or a power line communication (PLC) module). A corresponding one of these communication modules may communicate with the external electronic device via the first network 698 (e.g., a short-range communication network, such as BLUETOOTH™, wireless-fidelity (Wi-Fi) direct, or a standard of the Infrared Data Association (IrDA)) or the second network 699 (e.g., a long-range communication network, such as a cellular network, the Internet, or a computer network (e.g., LAN or wide area network (WAN)). These various types of communication modules may be implemented as a single component (e.g., a single IC), or may be implemented as multiple components (e.g., multiple ICs) that are separate from each other. The wireless communication module 692 may identify and authenticate the electronic device 601 in a communication network, such as the first network 698 or the second network 699, using subscriber information (e.g., international mobile subscriber identity (IMSI)) stored in the subscriber identification module 696.

The antenna module 697 may transmit or receive a signal or power to or from the outside (e.g., the external electronic device) of the electronic device 601. The antenna module 697 may include one or more antennas, and, therefrom, at least one antenna appropriate for a communication scheme used in the communication network, such as the first network 698 or the second network 699, may be selected, for example, by the communication module 690 (e.g., the wireless communication module 692). The signal or the power may then be transmitted or received between the communication module 690 and the external electronic device via the selected at least one antenna.

Commands or data may be transmitted or received between the electronic device 601 and the external electronic device 604 via the server 608 coupled with the second network 699. Each of the electronic devices 602 and 604 may be a device of a same type as, or a different type, from the electronic device 601. For example, the electronic devices 602 and 604 may be sensors or devices including sensors (e.g., biomolecular sensors) for providing sensor data to the electronic device 601 or the server 608. Additionally, the server 608 may develop the algorithm, e.g., as illustrated in FIG. 2 or 4, and utilize the algorithm to provide MH output to the electronic device 601.

All or some of operations to be executed at the electronic device 601 may be executed at one or more of the external electronic devices 602, 604, or 608. For example, if the electronic device 601 should perform a function or a service automatically, or in response to a request from a user or another device, the electronic device 601, instead of, or in addition to, executing the function or the service, may request the one or more external electronic devices to perform at least part of the function or the service. The one or more external electronic devices receiving the request may perform the at least part of the function or the service requested, or an additional function or an additional service related to the request and transfer an outcome of the performing to the electronic device 601. The electronic device 601 may provide the outcome, with or without further processing of the outcome, as at least part of a reply to the request. To that end, a cloud computing, distributed computing, or client-server computing technology may be used, for example.

Embodiments of the subject matter and the operations described in this specification may be implemented in digital electronic circuitry, or in computer software, firmware, or hardware, including the structures disclosed in this specification and their structural equivalents, or in combinations of one or more of them. Embodiments of the subject matter described in this specification may be implemented as one or more computer programs, i.e., one or more modules of computer-program instructions, encoded on computer-storage medium for execution by, or to control the operation of data-processing apparatus. Alternatively or additionally, the program instructions can be encoded on an artificially-generated propagated signal, e.g., a machine-generated electrical, optical, or electromagnetic signal, which is generated to encode information for transmission to suitable receiver apparatus for execution by a data processing apparatus. A computer-storage medium can be, or be included in, a computer-readable storage device, a computer-readable storage substrate, a random or serial-access memory array or device, or a combination thereof. Moreover, while a computer-storage medium is not a propagated signal, a computer-storage medium may be a source or destination of computer-program instructions encoded in an artificially-generated propagated signal. The computer-storage medium can also be, or be included in, one or more separate physical components or media (e.g., multiple CDs, disks, or other storage devices). Additionally, the operations described in this specification may be implemented as operations performed by a data-processing apparatus on data stored on one or more computer-readable storage devices or received from other sources.

While this specification may contain many specific implementation details, the implementation details should not be construed as limitations on the scope of any claimed subject matter, but rather be construed as descriptions of features specific to particular embodiments. Certain features that are described in this specification in the context of separate embodiments may also be implemented in combination in a single embodiment. Conversely, various features that are described in the context of a single embodiment may also be implemented in multiple embodiments separately or in any suitable subcombination. Moreover, although features may be described above as acting in certain combinations and even initially claimed as such, one or more features from a claimed combination may in some cases be excised from the combination, and the claimed combination may be directed to a subcombination or variation of a subcombination.

Similarly, while operations are depicted in the drawings in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system components in the embodiments described above should not be understood as requiring such separation in all embodiments, and it should be understood that the described program components and systems can generally be integrated together in a single software product or packaged into multiple software products.

Thus, particular embodiments of the subject matter have been described herein. Other embodiments are within the scope of the following claims. In some cases, the actions set forth in the claims may be performed in a different order and still achieve desirable results. Additionally, the processes depicted in the accompanying figures do not necessarily require the particular order shown, or sequential order, to achieve desirable results. In certain implementations, multitasking and parallel processing may be advantageous.

As will be recognized by those skilled in the art, the innovative concepts described herein may be modified and varied over a wide range of applications. Accordingly, the scope of claimed subject matter should not be limited to any of the specific exemplary teachings discussed above, but is instead defined by the following claims.

What is claimed is:

1. A method of metabolic sensing, the method comprising:
receiving first sensor data from a first sensor worn by a user;
receiving second sensor data from a second sensor worn by the user, the second sensor being a different type of sensor than the first sensor;
generating based at least in part on the first sensor data and the second sensor data, a metabolic health (MH) indicator, the MH indicator corresponding to the first sensor data, the second sensor data, and third sensor data of a third sensor that is different from the first and second sensors; and
providing the MH indicator to the user.

2. The method of claim 1, wherein the first sensor or the second sensor includes at least one of a photoplethysmography (PPG) sensor, an inertial measurement unit (IMU), an electrocardiogram (ECG) sensor, a multispectral sensor, a polarization sensor, a depth/3-dimensional (3D) sensor, or a temperature sensor.

3. The method of claim 1, wherein the third sensor includes at least one of a biomolecular sensor, an electrochemical sensor, a piezoelectric sensor, a field-effect transistor (FET) sensor, an immunosensor, or a nanomaterial-based sensor.

4. The method of claim 1, wherein the first sensor and the second sensor are included in a wearable device.

5. The method of claim 1, wherein the algorithm utilizes neural network-based machine learning tools to generate the MH indicator.

6. The method of claim 1, wherein the MH indicator includes at least one of a metabolic rate (MR), a metabolic fitness (MF) value, or a metabolic biomarker (MB).

7. The method of claim 6, wherein the MR includes at least one of a basal metabolic rate (BMR) or an active metabolic rate (AMR).

8. The method of claim 6, wherein the MF value includes at least one of a metabolic age or a glucose variation index (GVI).

9. The method of claim 6, wherein the MB includes a blood level indicator of at least one of glucose, lactate, triglycerides, or insulin.

10. The method of claim 1, wherein the first sensor data or the second sensor data includes at least one of heart rate (HR), HR variability (HRV), oxygen saturation (SpO2), steps taken, activity, skin temperature, or core body temperature.

11. The method of claim 1, wherein the third sensor data includes at least one of a glucose level, a lactate level, a triglycerides level, or an insulin level.

12. The method of claim 1, further comprising generating the algorithm for outputting the MH indicator using previous first sensor data, previous second sensor data, and previous third sensor data collected from one or more users over a training period.

13. The method of claim 12, wherein generating the algorithm for outputting the MH indicator further comprises:
selecting a neural network model based on the previous first sensor data, the previous second sensor data, and the previous third sensor data; and
training the neural network model using at least one of clinical laboratory tests or commercial sensor derived health data as ground truth data.

14. The method of claim 13, wherein the neural network model includes at least one of a convolutional neural network (CNN), a recurrent neural network (RNN), a transformer-based architecture, an autoencoder, a self-supervised learning model, or a reinforcement learning model.

15. A system for metabolic sensing, the system comprising:
a first sensor worn by a user;
a second sensor worn by the user, the second sensor being a different type of sensor than the first sensor; and
a processor configured to:
receive first sensor data from the first sensor,
receive second sensor data from the second sensor,
generate based at least in part on the first sensor data and the second sensor data, a metabolic health (MH) indicator, the MH indicator corresponding to the first sensor data, the second sensor data, and third sensor data of a third sensor that is different from the first and second sensors, and
provide the MH indicator to the user.

16. The system of claim 15, wherein the first sensor or the second sensor includes at least one of a photoplethysmography (PPG) sensor, an inertial measurement unit (IMU), an electrocardiogram (ECG) sensor, a multispectral sensor, a polarization sensor, a depth/3-dimensional (3D) sensor, or a temperature sensor.

17. The system of claim 15, wherein the third sensor includes at least one of a biomolecular sensor, an electrochemical sensor, a piezoelectric sensor, a field-effect transistor (FET) sensor, an immunosensor, or a nanomaterial-based sensor.

18. The system of claim 15, wherein the first sensor and the second sensor are included in a wearable device.

19. The system of claim 15, wherein the algorithm utilizes neural network-based machine learning tools to generate the MH indicator.

20. The system of claim 15, wherein the MH indicator includes at least one of a metabolic rate (MR), a metabolic fitness (MF) value, or a metabolic biomarker (MB).
